(19) United States
(12) Patent Application Publication
(10) Pub. No.: US 2026/0294525 A1
(43) Pub. Date: Oct. 1, 2026

(54) SELF-LOCKING, BI-STABLE, CRANK-BASED PULLWIRE ACTUATOR FOR INTRALUMINAL DEVICE

(71) Applicant: KONINKLIJKE PHILIPS N.V., EINDHOVEN (NL)

(72) Inventor: Mikhail Borisovich TIKH, ST.LOUIS PARK, MN (US)

(21) Appl. No.: 19/443,528

(22) Filed: Jan. 8, 2026

Related U.S. Application Data
(60) Provisional application No. 63/779,442, filed on Mar. 28, 2025.

(30) Foreign Application Priority Data
Jun. 27, 2025 (EP) 25185778.5

(51) Int. Cl. A61B 18/14; A61B 34/00; A61B 17/00; A61M 25/01

(52) U.S. Cl. CPC A61B 18/1492; A61B 34/71; A61B 2017/00323; A61B 2034/715; A61M 25/0136; A61M 25/0147

(57) ABSTRACT

An apparatus includes an intraluminal device. The intraluminal device includes a flexible elongate shaft that is positioned within a body lumen of a patient and a pullwire disposed within the flexible elongate shaft that transitions a distal portion of the flexible elongate shaft between a first configuration and a second configuration. The intraluminal device also includes a handle coupled to a proximal portion of the flexible elongate shaft. The handle includes an actuation mechanism connected to the pullwire. The actuation mechanism transitions the distal portion between the first configuration and the second configuration, maintains the distal portion in the first configuration or in the second configuration without continuous input by a user.

### TECHNICAL FIELD

The present disclosure relates to pullwire actuation mechanisms for intraluminal medical devices, and more particularly to a crank-based actuation mechanism that is self-locking and bi-stable. For example, the present disclosure allows for reduction in the amount of mechanical force required for user to operate an intraluminal device (e.g., heart valve leaflet modification device), such as transitioning a distal portion of the intraluminal device between a straight configuration and a hooked configuration.

### BACKGROUND

Many structural heart procedures, including valve replacement procedures, benefit from modification of the leaflets of native and/or implanted valves. For example, aortic valve replacement can cause the coronary vessels to become occluded when the leaflets of the native valve (or in the case of a valve-in-valve implant, the implanted valve leaflet), are pressed up against the side wall of the aortic valve annular region. Similarly, in a mitral valve replacement, the left ventricular out flow tract (LVOT) can become narrowed by the leaflets of the native valve or previously implanted valve. Another issue can be access to the coronary arteries after implantation of a prosthetic aortic valve. The leaflet of the native or implanted valve can prevent easy access for a guidewire to cross from the coronary sinus area into the ostia of the coronary arteries.

Intraluminal devices, such as those for heart valve leaflet modification, may include a pullwire to change the shape of the distal end of the device. The relatively large amount of force required to operate the pullwire, which may be challenging to apply and maintain in a surgical setting. The pullwire may also require a relatively large amount of travel to fully activate the intraluminal device. Directly pulling on the pullwire at the handle may be impractical and the need to hold the intraluminal device in particular configuration during a procedure.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Aspects of the present disclosure provide a pullwire actuation mechanism within a handle of an intraluminal device (e.g., an intracardiac catheter) that allowable high-force, extended-travel pullwire activation with minimal user effort. The handle incorporates a dual crank-slider mechanism with self-locking and bi-stable properties. The self-locking feature allows the actuation mechanism to maintain its position without continuous user input. The bi-stable feature allows the actuation mechanism to smoothly/naturally settle into one of two different states, which corresponds to two different shapes of the intraluminal device’s distal portion. A counterbalance spring offsets the pullwire force, significantly reducing the effort required of the user for activation. The over-center design of each crank-slider mechanism creates the self-locking feature, preventing unintended release. The combination of features described herein allows for precise control of the intraluminal device’s distal portion, e.g., allowing the intraluminal device’s distal portion to transition between two differently-shaped configurations with ease and stability.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1 is a top side perspective view of a leaflet puncture and slitting device, according to aspects of the present disclosure.

FIG. 2 is a side view of a distal portion of an example distal portion of a leaflet puncture and slitting device in its straight or piercing configuration, according to aspects of the present disclosure.

FIG. 3 is a side cross-sectional view of the distal portion of FIG. 2, according to aspects of the present disclosure.

FIG. 4 is a side view of the distal portion of the leaflet puncture and slitting device of FIG. 2 in the hooked configuration, according to aspects of the present disclosure.

FIG. 5 is a side cross-sectional view of distal portion of the leaflet puncture and slitting device of FIG. 4 in the hooked configuration, according to aspects of the present disclosure.

FIG. 6 is a close-up view of the handle of the leaflet puncture and slitting device 100 of FIG. 1 in the straight configuration or piercing configuration, according to aspects of the present disclosure.

FIG. 7 is a side cross-sectional view of the handle of FIG. 6 in the straight or piercing configuration, according to aspects of the present disclosure.

FIG. 8 is a side cross-sectional view of the handle of FIG. 7, but in the hooked or cutting configuration, according to aspects of the present disclosure.

FIG. 9 is a close-up, side cross-sectional view of the crank assembly of a leaflet puncture and slitting device in the straight or piercing configuration, according to aspects of the present disclosure.

FIG. 10 is a close-up, side cross-sectional view of the crank assembly of a leaflet puncture and slitting device in the hooked or cutting configuration, according to aspects of the present disclosure.

FIG. 11 is a schematic, diagrammatic, side view of the crank assembly of a leaflet puncture and slitting device, showing a bi-stable configuration, according to aspects of the present disclosure.

FIG. 12 is a schematic, diagrammatic, side-view representation of a self-locking pullwire actuator, according to aspects of the present disclosure.

FIG. 13 is a graphical representation of the forces acting on a self-locking pullwire actuator, according to aspects of the present disclosure.

FIG. 14 is a schematic, diagrammatic representation, in block diagram form, of an example leaflet puncture and slitting device that incorporates a self-locking pullwire actuator, according to aspects of the present disclosure.

### DETAILED DESCRIPTION

Disclosed herein is a self-locking pullwire actuator for a leaflet puncture and slitting device, with associated systems and methods. During a heart valve replacement or other intracardiac procedure, slitting a heart valve leaflet from the center of the valve annulus towards the root of the leaflet (or vice versa) can allow for blood flow and/or passage of a guidewire. A leaflet puncture and slitting device may, for example, include a first electrically-activatable electrode capable of puncturing the tissue of the leaflet in a straight configuration and a second electrically-activatable electrode which, in a hooked configuration, is capable of extending the puncture into a slit, from the base of the leaflet to its tip. The device is transitioned from the straight configuration to the hooked configuration when a tension force is applied to a pullwire. This leaflet puncture and slitting device enables a clinician to puncture the leaflet in a precise location and then slit it in a controlled manner.

Directly pulling on the pullwire at the handle is impractical due to the high force requirements, and using an industry standard twist style handle would be impractical due the many turns that would be required due to the extended pullwire travel. Therefore, in order to generate the approximately 15 lbf of pullwire force required to form and hold the hook shape at the distal end of the device, a self-locking pullwire actuation mechanism includes a crank with top and bottom links, where the top link is attached to the pullwire and the bottom link attached to a spring. Lbf can refer to pound(lb)-force(f), which is a unit of force in the Imperial system and represents the force exerted by gravity on a mass of one avoirdupois pound on Earth’s surface. The values described herein are exemplary and can include other suitable values (both larger and smaller). The values described herein include the stated value and/or other values within, e.g., ± 1%, ± 5%, or ± 10% of the stated value.

Provided herein is a pullwire actuation mechanism that carries a vast majority of the pullwire force required (e.g., approximately 13 lbf of the 15 lbf load). The links are arranged on the crank such that the position of the crank is bi-stable, e.g., it will hold itself in either the straight (piercing) configuration or the hooked (cutting) configuration, unless a force of approximately 2 lbf is applied to the crank lever.

The technical features of the present disclosure that address problems of high-force, extended-travel pullwire activation with minimal user input include:

Dual crank-slider mechanism utilizing a pair of balanced crank-slider mechanisms working in tandem. The mirrored arrangement of the two mechanisms automatically biases to either end of motion. This prevents the user from mistakenly attempting to move or operate the device in an intermediate configuration (between the two intended configurations).

Self-locking over-center design: each mechanism is designed to go over-center at the end of its travel, creating a self-locking feature that prevents unintended release. This creates a bi-stable configuration (first stable configuration is straight/piercing configuration and second stable configuration is the hooked/cutting configuration).

Counterbalance spring: a spring in the reverse mechanism offsets the force on the pullwire, significantly reducing the effort required for user activation.

Design of the self-locking pullwire actuator is based on the insight that combining two opposing crank-slider mechanisms with an over-center design can create a bi-stable, self-locking system. This configuration, along with the counterbalance spring, allows for high force output and extended travel while minimizing user input force.

The mechanism of the self-locking pullwire actuator includes a lever-actuated, crank-slider mechanism that self-locks in the over-center position. This mechanism offers several advantages, including:

Mechanical advantage: as the pull progresses, the mechanical advantage at the lever increases. This means less user effort is required as the mechanism approaches maximum tension.

Self-locking feature: by designing the crank to go over-center at the end of travel, the system becomes self-locking. In this position, the applied force tends to keep turning the crank clockwise. To release the mechanism, a deliberate counter-clockwise force must be applied to the lever.

Even with the mechanical advantage of the over-center mechanism and large lever handle, without additional mechanical support, the user would need to apply an unreasonable amount of force to get the fully actuated position. Additionally, when the over-center position is released, the lever could dangerously and uncontrollably accelerate due to the high initial force on the reverse swing. To mitigate this, a mirrored version of the mechanism is added in the reverse rotation direction with a counterbalance spring that offsets the force on the pullwire. Although the force profiles are not identical, a counterbalance spring (selected to closely match the pullwire load in travel and spring rate) minimizes the force required for the user to actuate the pullwire. Furthermore, having this second over-center mechanism on the reverse stroke creates the bi-stable mechanism, which automatically biases to either end of motion with a tipping point approximately at the midpoint, as described below in further detail.

Aspects of the actuation mechanism can be implemented in any suitable intraluminal medical device, such as a catheter or other flexible, elongate device (e.g., for treatment of a patient body and/or sensing of intraluminal data). Examples of such devices include flexible, elongate intracardiac medical devices and/or flexible, elongate intracardiac medical devices intravascular medical devices, such as those for treatment delivery (e.g., ablation, such as for electrophysiology (EP)), and/or sensing (e.g., blood pressure sensor, blood flow sensor, imaging, such as ultrasound imaging or optical coherence tomography (OCT)).

One specific example is for a leaflet resection device in intracardiac procedures such as heart valve replacement. Aspects of the present disclosure improve a clinician’s ability to cut (e.g., bisect) a valve leaflet with precision, and with minimal application of force to actuate the pullwire. Implemented in the handle of a leaflet puncture and slitting device, the self-locking pullwire actuator disclosed herein provides practical, precise surgical capabilities in an intracardiac environment. This improved leaflet modification technique transforms a complex, potentially imprecise procedure requiring high levels of skill and training into a repeatably precise leaflet slitting technique, without the need to apply a force of approximately 15 lbf to the pullwire in order to actuate the device. This unconventional approach improves the functioning of the leaflet puncture and slitting device, by reducing the amount of force required to transition from the straight to the hooked configuration, and by holding (e.g., locking) the device in either the straight or the hooked configuration unless a force of approximately 2 lbf is applied to switch between configurations.

It is noted that the straight configuration and hooked configuration are two example shapes that are controlled by one or more pullwires. Aspects of the present disclosure contemplate any two differently-shaped configurations of a flexible elongate shaft/member that are controlled by one or more pullwires, e.g., straight, hooked, deflected, curved, circular, helical, combinations thereof, etc.

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to the heart and/or blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

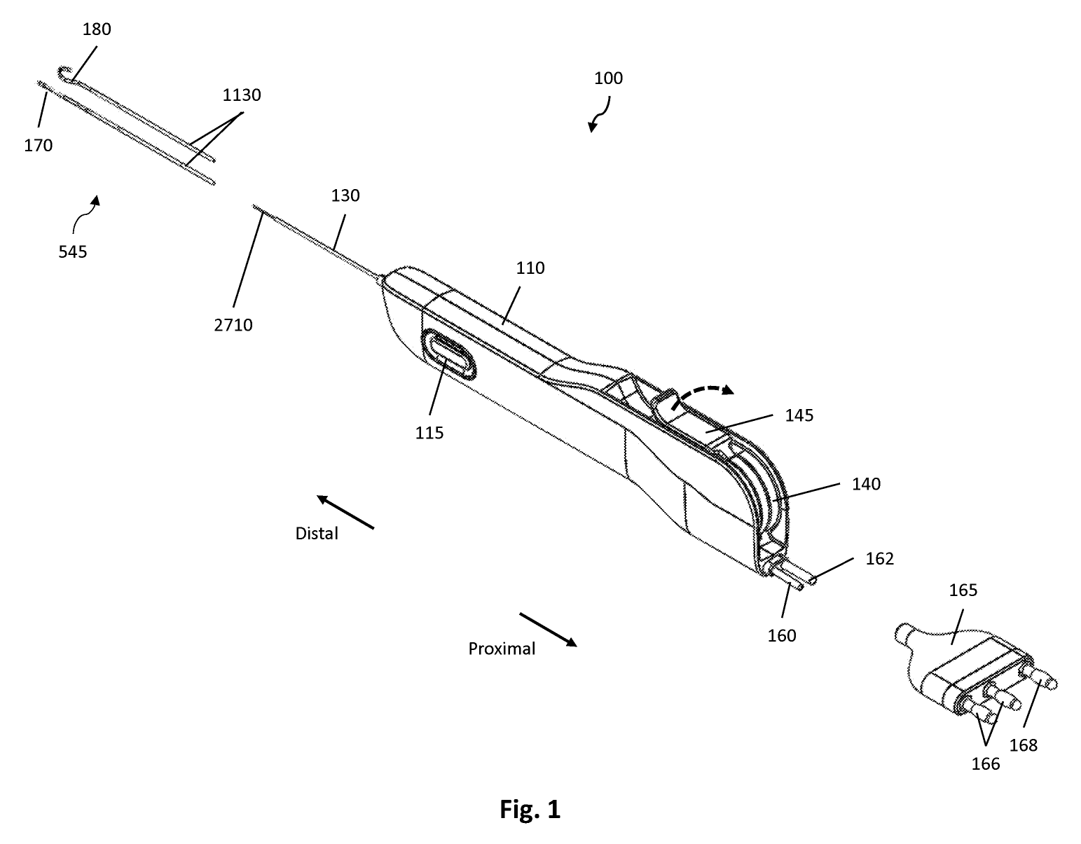

FIG. 1 is a perspective, partially-exploded view of a leaflet puncture and slitting device 100, according to aspects of the present disclosure. The leaflet puncture and slitting device 100 includes a handle 110 and a distal portion 545 including the piercing electrode and the cutting electrode. The handle 110 can be grasped within the hand of a user (e.g., a physician or other clinician). The distal portion 545 extends inside of the patient’s body. The distal portion 545 can be advanced through the patient’s blood vessels and into a heart of the patient.

Because FIG. 1 is a partially exploded view, it is understood objects that are shown as separated in FIG. 1 are actually coupled to one another in the final assembled form of the device that is used during a procedure on a patient. The distal portion 545 includes hypotube 1130, which can be transitioned between two different shaped configurations (e.g., straight configuration 170 and hook shaped configuration 180). FIG. 1 shows two copies of the hypotube 1130 to illustrate the differentially shaped configurations, but it is understood that the device 100 only includes one hypotube. The hypotube 1130 can be cutting electrode. In some aspects, the piercing electrode can be an electrode that is coupled at the distal end of the hypotube 1130.

The device 100 additionally includes the flexible elongate shaft 130 and a pullwire 2710 (e.g., pullwire conductor) extending within the flexible elongate shaft 130. The pullwire conductor 2710 can be mechanically and electrically coupled to the piercing electrode. In some aspects, the hypotube 1130 can be part of the flexible elongate shaft 130. For example the flexible elongate shaft 130 can include the hypotube 1130 and a polymer/plastic portion that surrounds a portion of the hypotube 1130. An uncovered portion of the hypotube 1130 can be the cutting electrode.

The handle 110 includes an electrode activation button 115, as well as a crank 140 and crank lever 145 of the self-locking pullwire actuator, for actuating the pullwire 2710. The handle 110 also includes an electrical connection 160 and a fluid lumen 162. The electrical connection 160 carries electrical power to the cutting electrode and the piercing electrode, and connects to an RF electrical connector 165, which includes electrode power connections 166 and a ground connection 168. The fluid lumen 162 can be for delivery of fluid into the patient body during the procedure, such as water, dextrose, and/or saline.

When the leaflet puncture and slitting device 100 is at rest, with the crank lever in the front position (shown in FIG. 1), the distal portion 545 is in a straight (e.g., piercing) configuration 170. However, when the crank lever 145 is pulled into the back position (indicated by the arrow in FIG. 1) so as to rotate the crank 140, the crank 140 pulls the pullwire 2710, which transitions the distal portion 545 from the straight configuration 170 to a hooked (e.g., cutting) configuration 180.

The handle 110, button 115, crank 140, and/or crank handle 145 may be made of any suitable material, including but not limited to polymers such as acrylonitrile butadiene styrene (ABS), polycarbonate (PC), ABS-PC blend, nylon, etc. The flexible elongate shaft, hypotube, and/or pullwire may be made of any suitable material, including but not limited to shape memory alloys such as nitinol.

It is noted that the self-locking pullwire actuator can be applied to other types of intraluminal devices besides leaflet puncture and slitting devices, including, without limitation, imaging devices, ablation devices, cryotherapy devices, or any other device that includes one or more high-tension, high-travel pullwires. However, for ease of explanation within this disclosure, many examples will refer to leaflet puncture and slitting device.

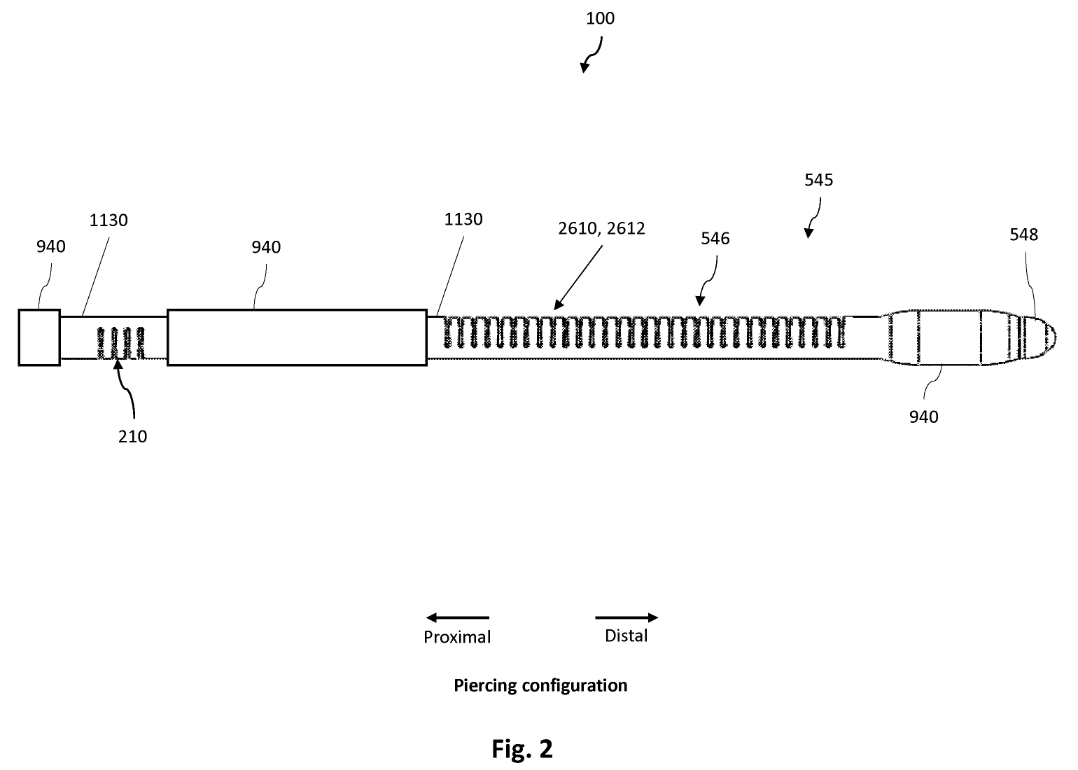

FIG. 2 is a side view of a distal portion of an example distal portion 545 of a leaflet puncture and slitting device in its straight or piercing configuration, according to aspects of the present disclosure. Visible are the cutting electrode 546 and the crossing electrode 548. The crossing electrode 548 can also be referred to as a piercing electrode. The intended anatomical targets for the cutting electrode and the crossing electrode 548 include structural heart anatomy, such as aortic valve leaflets.

The crossing electrode 548 is positioned at, coupled to, forms, and/or otherwise defines the distal tip of the distal portion 545. The crossing electrode 548 is made of a conductive metal or metal alloy. The distal portion of the crossing electrode 548 is uncovered and exposed to the patient’s anatomy. In the straight configuration shown in FIG. 2, the crossing electrode 548 can be energized and contacted with the heart valve leaflet to pierce the leaflet. Accordingly, the straight configuration of FIG. 2 can also be referred to a piercing configuration or a crossing configuration.

The distal portion 545 includes the hypotube 1130. The hypotube 1130 can also be referred to as a conductive hypotube 1130 because, as described herein, the hypotube 1130 can carry electrical energy. In some examples, the hypotube 1130 extends for a majority of the length of the leaflet puncture and slitting device, such as between the proximal portion and the distal portion 545. The hypotube 1130 can include a cut portion 2610. In some aspects, the entire length of the hypotube 1130 is the cut portion 2610. In other example, the hypotube 1130 includes both one or multiple cut portions 2610 and one or multiple uncut portions.

The cutting electrode 546 is formed by, includes, and/or is otherwise defined by the cut portion 2610 of the hypotube 1130. The cut portion 2610 includes cuts 2612 between segments of the hypotube 1130. The cuts 2612 can also be referred to as spaces or gaps. The cut portion 2610 can be cut by laser cutting other suitable types of cutting, such as wire electrical discharge machining (wire EDM). While the portion 2610 is referred to as a cut portion 2610, in other aspects, other types of manufacturing (e.g., 3D printing), are used to form the portion 2610 with the spaces 2612.

The cutting electrode 546 is made of a conductive metal or metal alloy. The cutting electrode 546 is uncovered and exposed to the patient’s anatomy. However, when piercing or crossing the leaflet in the straight configuration of FIG. 2, electrical energy is not delivered to the hypotube 1130 (and thus the cutting electrode 546), such that the cutting electrode 546 is not energized to cut the leaflet. As described in more detail herein, the cutting electrode 546 is energized when the distal portion 545 is in the hooked configuration for cutting the leaflet.

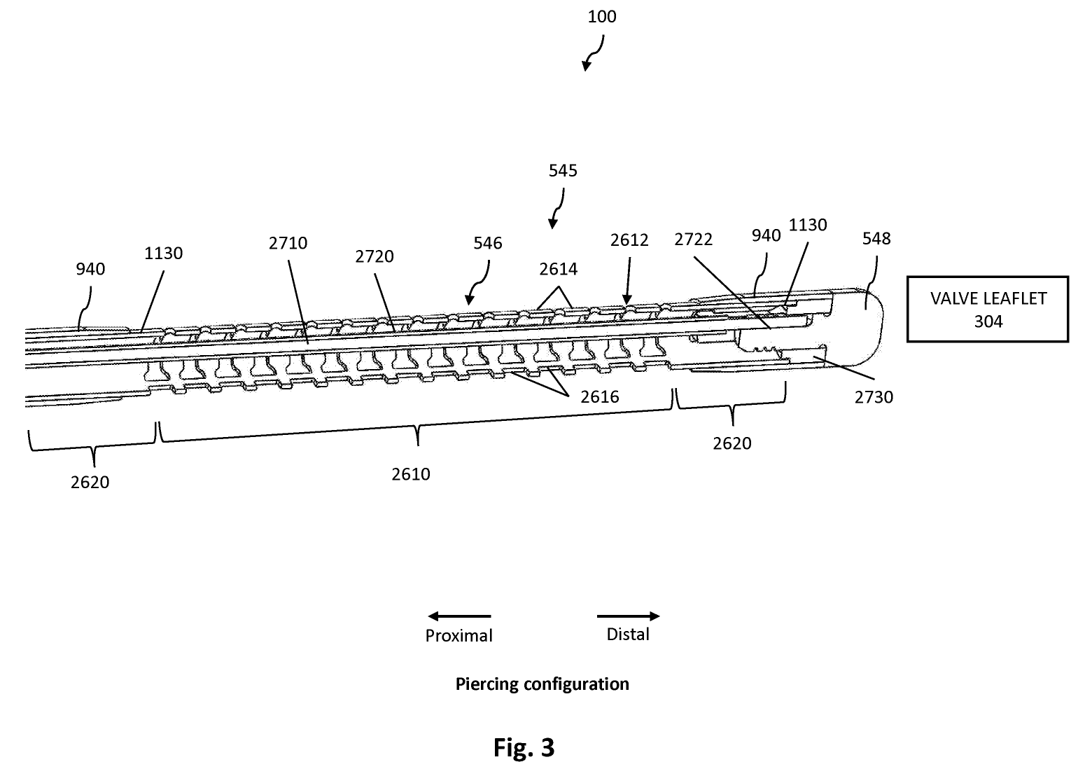

The distal portion 545 includes different sections of outer insulation layer 940. The outer insulation layer 940 can be one or multiple polymers that provide electrical isolation for the crossing electrode 548 and/or the cutting electrode 546. One section of the outer insulation layer 940 is located proximal of the crossing electrode 548 and the distal of the cutting electrode 546 (e.g., between the crossing electrode 548 and the cutting electrode 546). This section of the outer insulation layer 940 can surround a proximal end of the crossing electrode 548 and the electrical/mechanical connections between a pullwire conductor 2710 and the crossing electrode 548 (shown and described below with respect to FIG. 3). Another section of the outer insulation layer 940 is located proximal of the cutting electrode 546. This portion of the outer insulation layer 940 surrounds the portion of the hypotube 1130 that is proximal of the cut portion 2610 such that cutting is only performed by the cutting electrode 546 when the hypotube 1130 is energized.

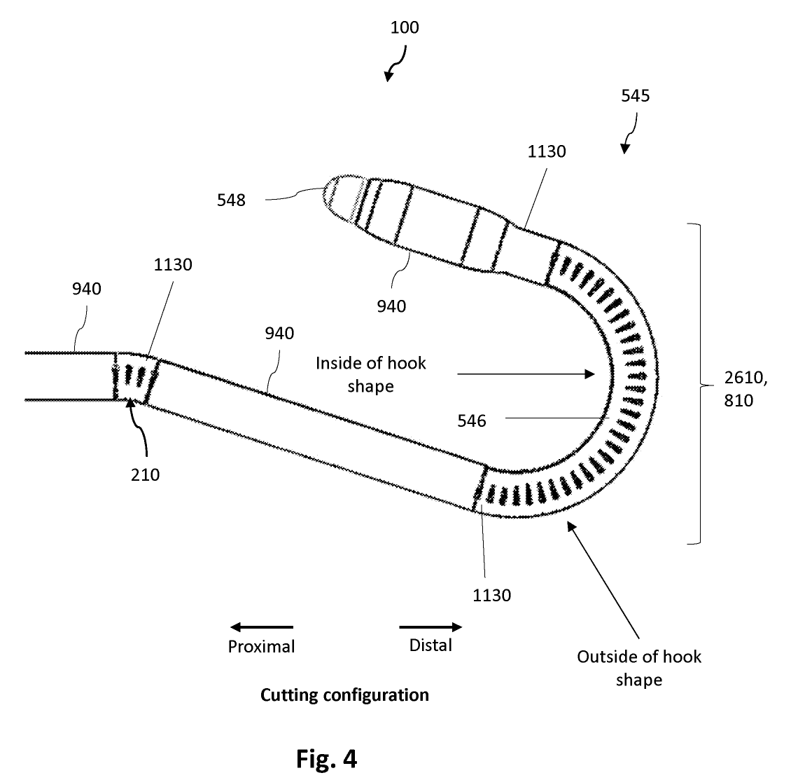

Also visible is a second cut portion 210 of the hypotube 1130, which allows deflection of the elongate shaft, as shown in FIG. 4.

FIG. 3 is a side cross-sectional view of the distal portion 545 of FIG. 2, according to aspects of the present disclosure. Visible are the outer insulation layer 940, cutting electrode 546, crossing electrode 548, hypotube 1130, laser-cut section 2610, and uncut sections 2620.

Also visible is a conductive pullwire or pullwire conductor 2710 surrounded by an inner layer of insulation 2720. In the example shown in FIG. 3, the distal end of the conductive pullwire 2710 (a portion not surrounded by the inner layer of insulation 2720) is electrically coupled and mechanically coupled (e.g., welded, soldered, adhered with a conductive adhesive, etc.) directly to the proximal end of crossing electrode 548 at a location or length 2722. The crossing electrode 548 is electrically isolated from the hypotube 1130 by an insulator 2730, but is electrically connected to the pullwire 2710, which acts as an electrical conductor to energize the crossing electrode 548, for piercing of the heart valve leaflet 304.

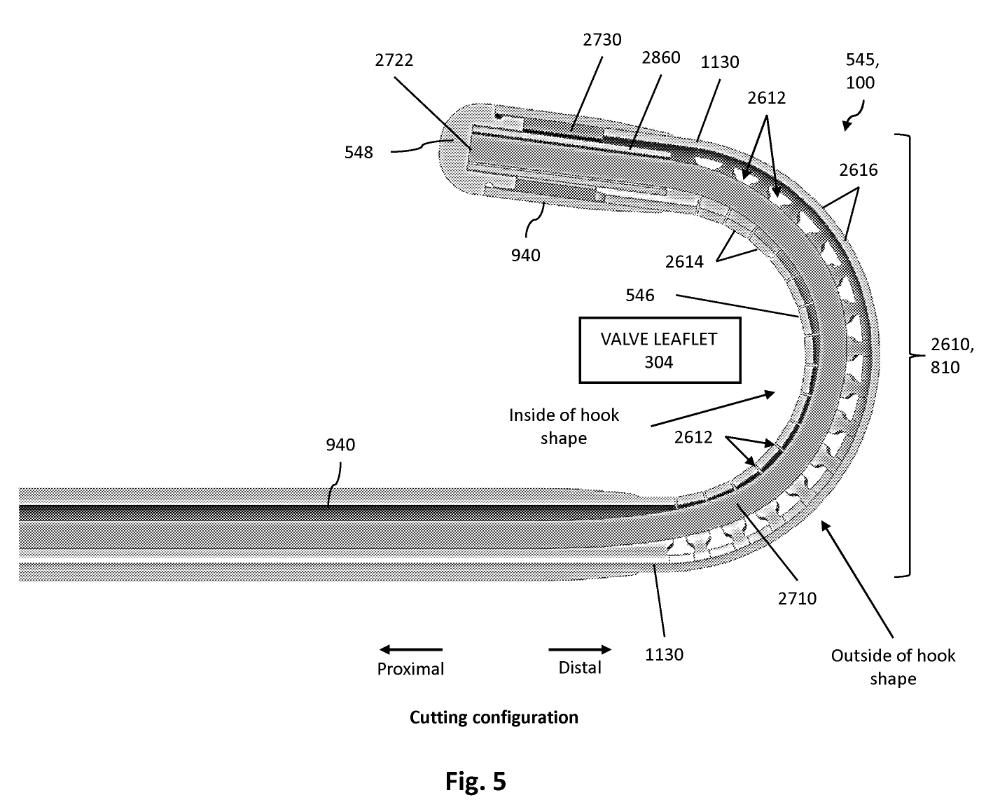

The cross-sectional view of the cut portion 2610 of the hypotube 1130 includes multiple segments 2614 that are separated by gaps 2612. The gaps 2612 allow the segments 2614 of the cut portion 2610 to be angled relative to another (in response to tension applied to the pullwire conductor 2710 when pulled) so that the cutting electrode 546 transitions from the straight configuration (e.g., FIGS. 2 and 3) to the hook-shaped configuration (e.g., FIGS. 4 and 5). The cut portion 2610 also has connectors 2616, which are uncut segments of the hypotube 1130. The connectors 2616 physically connect the segments 2614 to one another. The physical continuity provided by the connectors 2616 to the cut portion 2610 allow for all of the cutting electrode 546 to be energized, such as when the hypotube 1130 as a whole is energized. In the example shown in FIG. 3, all of the connectors 2616 are located on one side of the resection device 545 (bottom in drawing). As shown and described with respect to FIGS. 4 and 5, the connectors 2616 are positioned on the outer portion when the cutting electrode 546 is in the hook-shaped configuration.

In other examples, the distal portion 545 can include a catheter shaft with a dedicated lumen for the pullwire 2710. The crossing electrode 548 can be bonded to such a catheter shaft.

FIG. 4 is a side view of the distal portion 545 of the leaflet puncture and slitting device of FIG. 2 in the hooked configuration, according to aspects of the present disclosure. Visible are the outer insulation layer 940, cutting electrode 546, crossing electrode 548, and hypotube 1130. After the crossing electrode 548 has pierced the valve leaflet, proximal tension on the pullwire (when the pullwire is pulled in the proximal direction) has pulled the cut section 2610 into a hook shape 810. The cutting electrode 546 can be pulled through the valve leaflet in order to slit or resect it. In particular, the portion of the valve leaflet that is proximate to the inside of the hook shape 810 is slit when the resection device 545 is pulled in the proximal direction (causing the energized cutting electrode 546 to move in the proximal direction through this portion of the valve leaflet 304).

In the example shown in FIG. 4, the portion of the hypotube 1130 that includes the cutting electrode 546 is laser cut or otherwise cut, forming a cut section 2610 that is flexible in such a way that when actuated by the pullwire, the cut section 2610 reversibly bends or folds into a hook shape 810 that is rigid as long as tension is maintained on the pullwire. The crossing electrode 546 forms the top of the hook shape via the cut section 2610 and is proximal-facing in the hook-shaped configuration (compared to distal facing in the straight configuration of FIGS. 2 and 3). With the cutting electrode 546 in the hook-shaped configuration, the cutting electrode 546 can be energized and the resection device 545 can be pulled proximally in order to resect the leaflet. Once the resection is complete, tension on the pullwire may be released, thus allowing the device to return to its straight configuration. The cutting electrode 546 and/or the distal portion 545 transitions from the hook-shaped configuration to the straight configuration, and the device is extracted from the patient body.

The laser cut hypotube 1130 is advantageously flexible yet maintains other desirable properties of a hypotube (e.g., a rigid profile and good torque transmission). In some existing devices, structures at the distal end of a catheter are often created using nitinol super-elastic metal. The disadvantage of such structures is a need to carefully balance forces, between structures being too rigid to fit in a delivery sheath on the one hand, and on the other hand not being rigid enough to perform the desired function. An advantage of the laser-cut hypotube in that when it is activated by a pullwire, it curls into a hook shape of a desired radius. As long as sufficient tension is maintained on the pullwire, the resulting hook shape is rigid enough for tissue cutting application and other applications where a rigid hook shape may be desirable. The process is reversible, e.g. by releasing tension on the pullwire, at which time the cut hypotube can be easily straightened again for extraction.

Also visible is a second cut section 210 of the hypotube 1130, which allows deflection of the elongate shaft.

FIG. 5 is a side cross-sectional view of distal portion 545 of the leaflet puncture and slitting device of FIG. 4 in the hooked configuration, according to aspects of the present disclosure. Visible are the outer insulation layer 940, cutting electrode 546, crossing electrode 548, hypotube 1130, cut section 2610, and pullwire conductor 2710. The insulator 2730 electrically isolates the cutting electrode 546 and the crossing electrode 548 from one another. The pullwire conductor 2710 is attached to the crossing electrode 548 such that it can be used as a conductor to energize the crossing electrode 548, and such that when tension is applied to the pullwire conductor 2710, the cut section 2610 of the hypotube 1130 forms the hook shape 810, with the cutting electrode 546 on the inside of the hook shape.

In the example shown in FIG. 5, once the crossing electrode 548 has pierced the valve leaflet 304, tension on the pullwire conductor 2710 has pulled the laser-cut section 2610 into a hook shape 810, such that the cutting electrode 546 can be pulled through the valve leaflet 304 in order to slit or resect it, as described above.

In the example of FIG. 5, the leaflet puncture and slitting device 100 includes one pullwire conductor 2710. The one pullwire conductor 2710 can allow for one type of deflection for the distal portion of the resection device 545. In particular, the cutting electrode 546 and/or the resection device 545 deflects in one direction when tension is applied to the pullwire conductor 2710. As shown in FIG. 5, for example, a longitudinal axis of the pullwire conductor 2710 and/or the coupling location 2722 between the pullwire conductor 2710 and the crossing electrode 584 can be positioned so that it is radially offset from the central longitudinal axis of the resection device 545 and/or the hypotube 1130 (e.g., radially positioned towards the inside of the hook shape). The pullwire conductor 2710 and/or coupling location 2722 being radially offset allows for the deflect when tension is applied to the pullwire conductor 2710. As such, the cut section 2610 of the hypotube 1130 flexes readily to form the hook shape 810, but does not easily flex in the in the direction opposite the hook, or out of the plane of the hook, such that when tension is applied to the pullwire 270, the cut section 2610 flexes to form the hook shape 810 with the cutting electrode 546 on the inside of the hook shape, rather than an outside of the hook shape. The one direction of deflection results in, for example, the side of the cut potion 2610 with less spacing in the straight configuration (top side in FIGS. 2 and 3) becomes the inside of the hook shape 810, the side of the cut potion 2610 with more spacing in the straight configuration (bottom side in FIGS. 2 and 3) becomes the outside of the hook shape 810 side, and the connectors 2616 between segments 2614 are on the outside of the hook shape 810. The cross-sectional view plane in FIG. 5 extends through the connectors 2616, which shows the physical continuity that that connectors 2616 provide to the cut portion 2610.

Deflection into the hooked-shaped configuration of the cutting electrode 546 and/or the distal portion 545 can cause the segments of the cut portion 2610 to be closer to one another on the inside of the hook shape 810 and farther away from one another on the outside of the hook shape 810. In some aspects, the edges of the segments on the inside of the hook shape 810 can be in contact with another in the hook-shaped configuration. In other aspects, the edges of the segments on the inside of the hook shape 810 are still spaced from one another in the hook-shaped configuration, but the spacing is less than in the straight configuration (FIGS. 2 and 3). The spacing between the segments 2614 in the hooked shaped configuration can depend on the amount of tension on the pullwire conductor 2710 (more tension results in less spacing, less tension results in more spacing). The connectors 2616 between segments 2614 are the outside of the hook shape 810.

In FIG. 5, the proximal end of crossing electrode 548 has an opening that receives the distal end of the pullwire conductor 2710. The pullwire conductor 2710 can be mechanically and electrically coupled to the crossing electrode 548 inside of the opening at the location 2722. An end hypotube 2860 is crimped, friction-fit, or soldered to the pullwire conductor 2710 and welded or soldered to the crossing electrode 548, to form both the electrical and mechanical connections between the pullwire conductor 2710 and the crossing electrode 548.

Further exemplary details about the leaflet puncture and slitting device 100 can be found in U.S. Provisional Application No. 63/527,871, filed Jul. 20, 2023, and titled “Multiple Electrode Heart Valve Slitting Device”, and International Application No. PCT/EP2024/069683, filed Jul. 11, 2024, titled “Multiple Electrode Heart Valve Slitting Device”, each of which is incorporated by reference as though fully set forth herein.

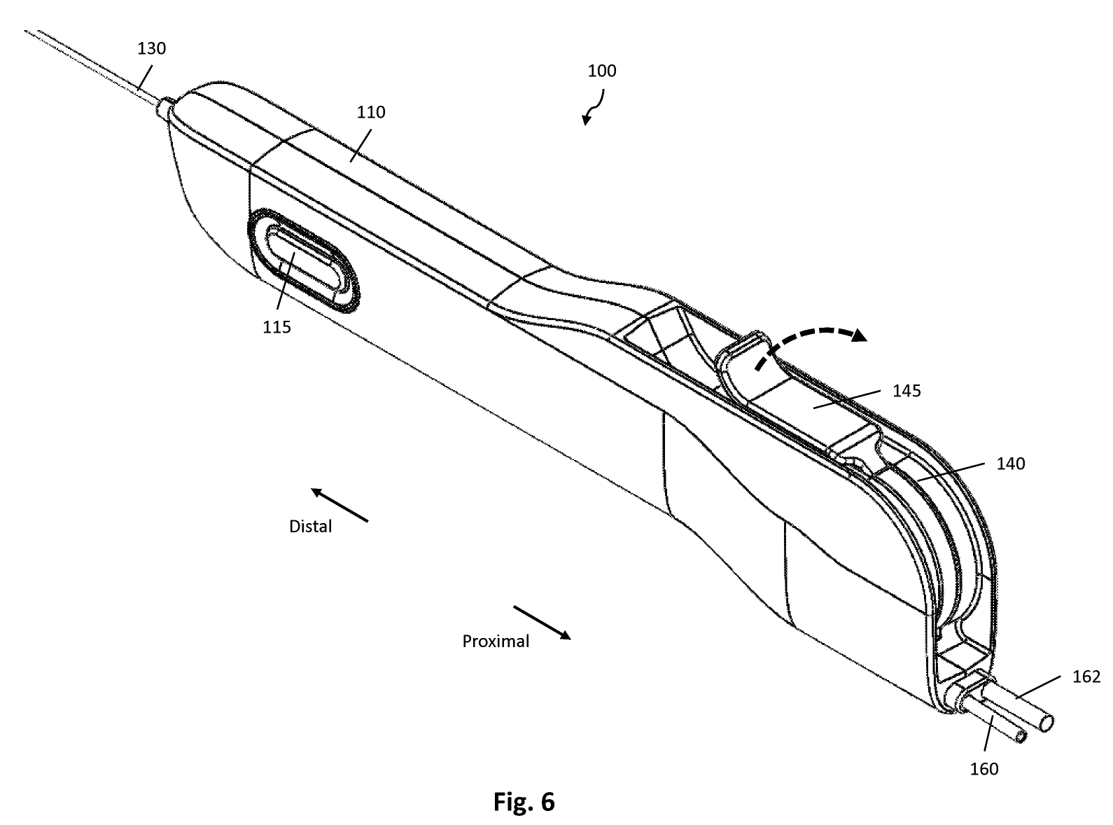

FIG. 6 is a close-up view of the handle 110 of the leaflet puncture and slitting device 100 of FIG. 1 in the straight configuration or piercing configuration, according to aspects of the present disclosure. Visible are the flexible elongate shaft 130, the electrode activation button 115, the crank 140, and the crank lever 145. While the user (e.g., a heart surgeon or other clinician) presses the electrode activation button 115 while the device is in the straight (piercing) configuration, the piercing electrode or crossing electrode is activated, and is then deactivated when the button 115 is released.

When the user pulls pack on the crank lever 145, the crank 140 is rotated, which pulls back on the pullwire and this transitions the distal portion of the leaflet puncture and slitting device 100 into its hooked configuration or cutting configuration. While the user presses the electrode activation button 115 while the device is in the hooked (cutting) configuration, the cutting electrode is activated, and is then deactivated when the button 115 is released. The electrical connection 160 can be include one or multiple wires providing electrical connection, such as to an RF generator source to provide current for the piercing electrode and/or the cutting electrode. The fluid lumen 162 allows delivery of fluid into the patient body via, e.g., the elongate shaft 130 and/or the cuts/kerfs in the hypotube 1130.

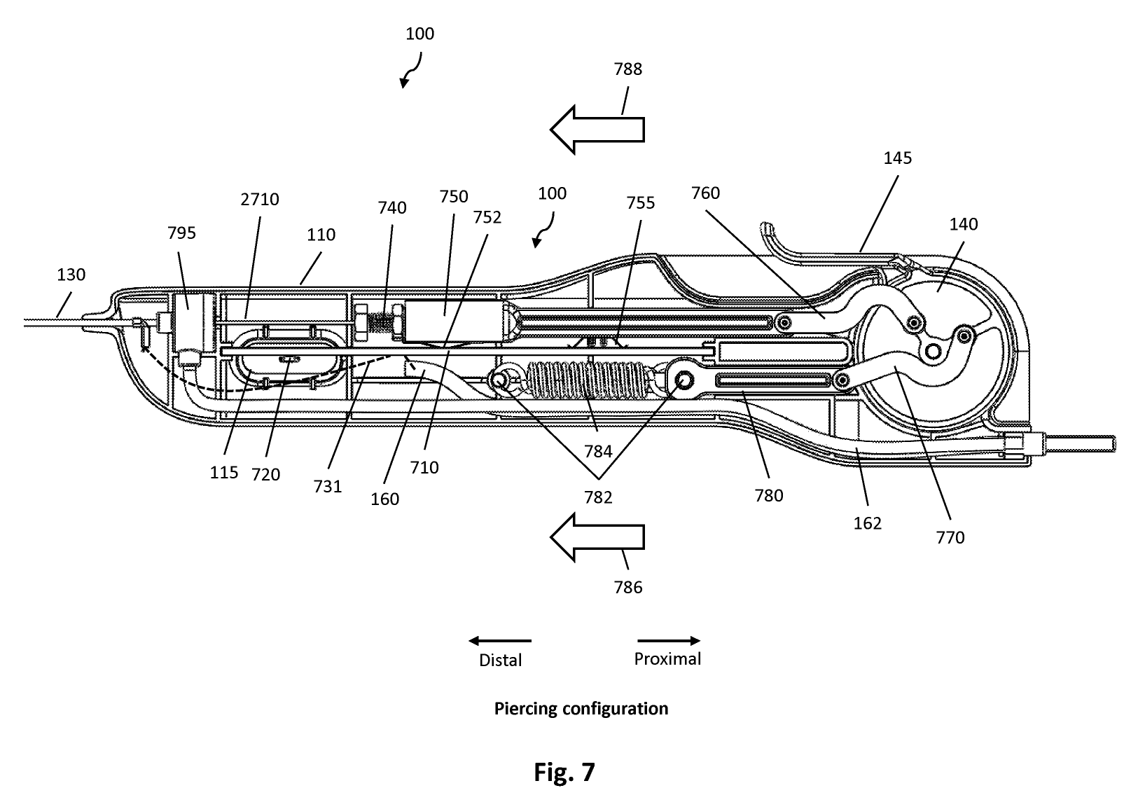

FIG. 7 is a side cross-sectional view of the handle 110 of FIG. 6 in the straight or piercing configuration, according to aspects of the present disclosure. Inside of the handle 110 are components forming an actuation mechanism (e.g., pullwire 2710, coupling 740, slider 750, link 760, crank 140, crank lever 145, spring 784, slider 780, and/or link 770) for transitioning the distal portion of the device between two differently-shaped configurations (e.g., straight/piercing configuration and hooked/cutting configuration). Visible are the flexible elongate shaft 130, the electrode activation button 115, the crank 140, and the crank lever 145. Also visible is a circuit board 710, which includes a contact 720 that is closed when the electrode activation button 115 is pressed, to energize the selected (e.g., piercing) electrode, which activates the current. Pressing the button 115 again, deactivates the current, which de-energizes the selected (e.g., piercing) electrode. An electrical cable 731 can provide electrical connection between the hypotube and the circuit board 710. Electrical cable 162 connects to the circuit board 710 and includes one or more wires that extend from the circuit board 710, out the proximal end of the handle and to the RF generator source.

The pullwire 2710 exits from the flexible elongate shaft 130 and is anchored to an adjustable coupling 740, which is fixedly attached to a top slider 750. The top slider 750 is rotatably coupled to the distal end of a top link 760, whose proximal end is rotatably coupled to the crank 140. A bottom link 770 also has a proximal end that is rotatably coupled to the crank 140. The distal end of the bottom link 770 is rotatably coupled to a bottom slider 780, which is coupled to a counterbalance spring 784. Pins 782 can provide coupling between the slider 780 and the spring 784 at the distal portion of the slider 780 and coupling between the slider 780 and the link 770 at the proximal portion of the slider 780.

The top slider 750 also includes contacts 755 that, in the straight configuration, make contact with the circuit board 710 (e.g., a contact pad) at a position that selects the piercing or crossing electrode, such that when the electrode activation button 115 is pressed, energizing the contact 720, the piercing electrode is energized. The top slider 750 also includes spring contact 750 that is connected electrically the pullwire conductor 2710 (via coupling 740) and connected to a contact pad on the circuit board 710.

The top slider 750 is shown in its most distal position, which equates to the straight (piercing) configuration for the distal portion of the leaflet puncture and slitting device. The spring 784 is therefore in an elongated configuration, and thus the spring 784 exerts a tensile force 786 in the distal direction, which thus tends to pull the bottom slider 780 in a distal direction, and thus rotate the crank 140 in a clockwise direction, adding tension to the pullwire 2710. However, the pullwire 2710 and/or components attached to the pullwire 2710 (which can modeled/considered as a spring) also exerts a tensile force 788 in the distal direction, which thus tends to pull the top slider 750 in a distal direction, and thus rotate the crank 140 in a counterclockwise direction. In some aspects, the self-locking pullwire actuator may be designed such that the tensile force 786 and the tensile force 788 approximately cancel one another, e.g., that that are within approximately 2 lbf of one another. In some aspects, 2 lbf can be a reasonable ergonomic force to expect a user to apply. Furthermore, locking features in the top link 760 and bottom link 770 cause the system to rest in either the straight or hooked configuration, and to transition towards whichever of these configurations is closest. These features will be discussed in more detail below.

Also visible is a fluid lumen 162 that carries fluid to a hemostatic valve 795, which is capable of dispensing the fluid down the flexible elongate shaft 130 to be released at the distal end of the leaflet puncture and slitting device. Such fluid may for example include a solution of water, dextrose, saline, or combinations thereof that flows out through the gaps in the cut portion of the hypotube and into the body lumen, thereby suppressing electrical conduction by blood proximate to the cutting electrode.

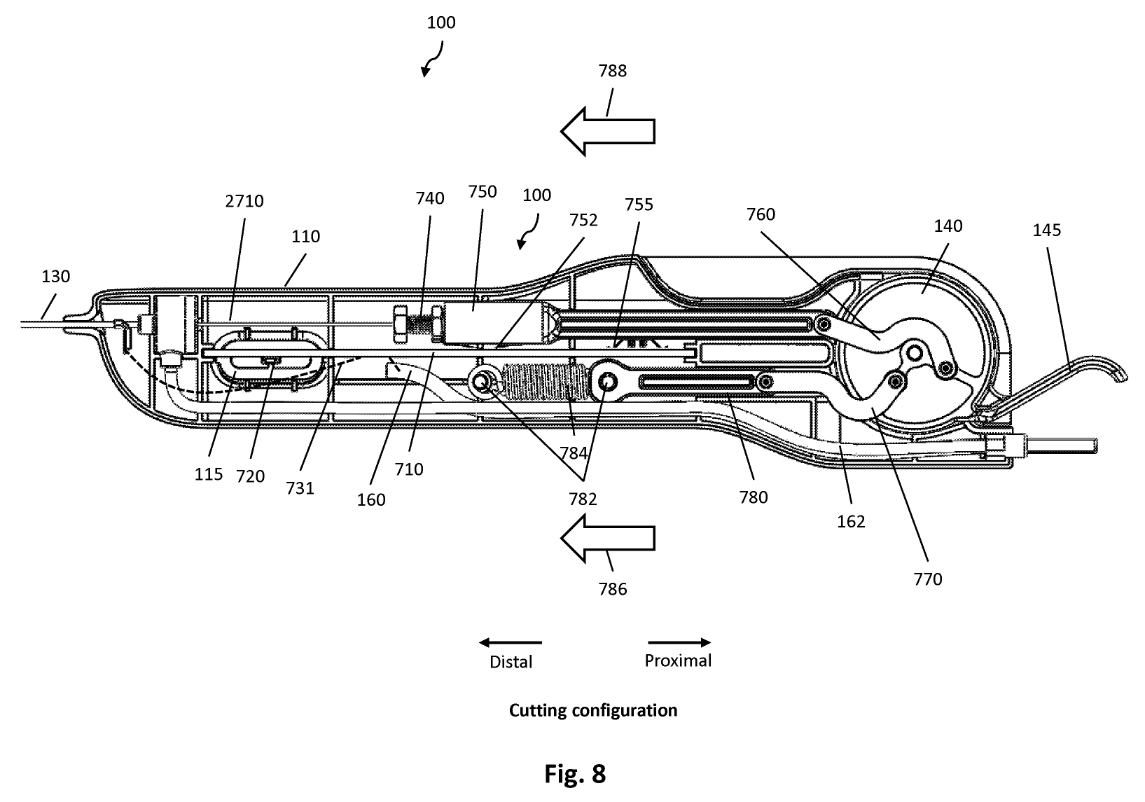

FIG. 8 is a side cross-sectional view of the handle 110 of FIG. 7, but in the hooked or cutting configuration, according to aspects of the present disclosure. Inside of the handle 110 are components forming an actuation mechanism ((e.g., pullwire 2710, coupling 740, slider 750, link 760, crank 140, crank lever 145, spring 784, slider 780, and/or link 770) for transitioning the distal portion of the device between two differently-shaped configurations (e.g., straight/piercing configuration and hooked/cutting configuration). Visible are the flexible elongate shaft 130, the electrode activation button 115, the crank 140, the crank lever 145, circuit board 710, and contact 720, which is closed when the electrode activation button 115 is pressed, to energize the selected (e.g., cutting) electrode. Also visible are the pullwire conductor 2710, adjustable coupling 740, top slider 750, top link 760, bottom link 770, bottom slider 780, pins 782, spring 784, spring contact 752, electrical connections 731 and 160, and fluid delivery lumen 162.

When the crank lever 145 is pulled backward (e.g., in a proximal direction), the crank 140 rotates in a clockwise direction such that the top link 760 is pulled in a proximal direction, which moves the top slider 750 to its most proximal position. The pullwire 2710, attached to the top slider 750 by the adjustable coupling 740, is pulled backward such that the distal portion of the leaflet puncture and slitting device is pulled into the hooked or cutting configuration. In addition, the contact 755 on the top slider 750, in the hooked or cutting configuration, make contact with the circuit board 710 (e.g., a contact pad) at a position that selects the cutting electrode, such that when the electrode activation button 115 is pressed, energizing the contact 720, the cutting electrode is energized rather than the piercing electrode.

The spring 784 is now in a relaxed state and exerts a reduced tensile force 786 in the distal direction, whereas the tensile force 788 exerted by the pullwire 2710 is increased in the hooked or cutting configuration. Thus, the crank 140 would tend to rotate in a counterclockwise direction if not for the locking features of the top link 760, described in further detail below. The magnitude of the forces 786, 788 can change as the crank is actuated.

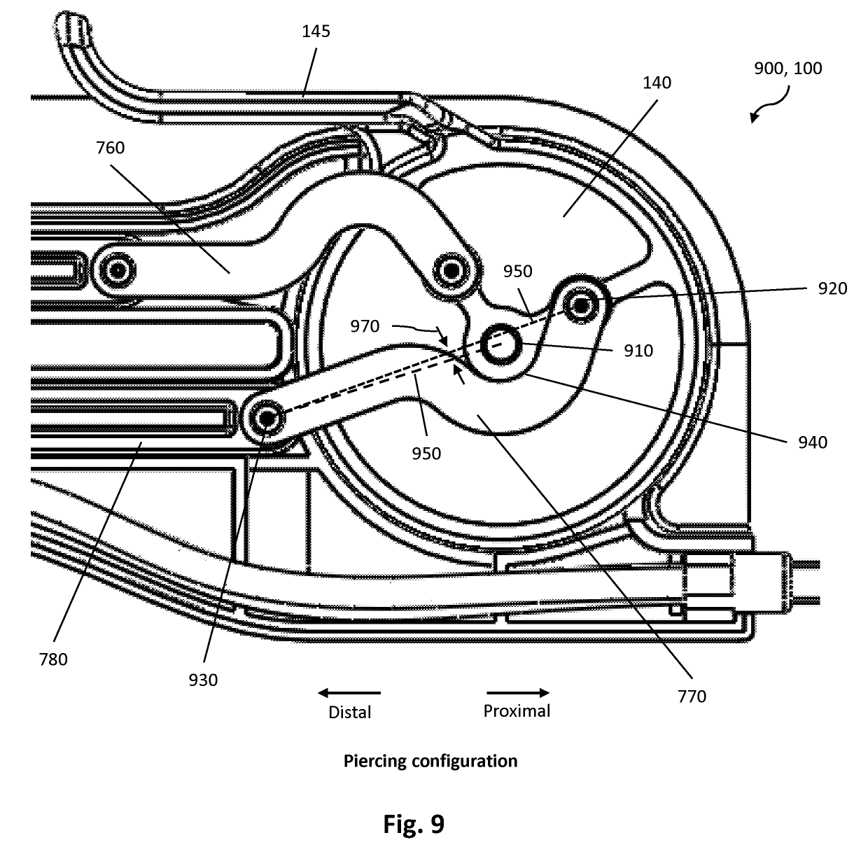

FIG. 9 is a close-up, side cross-sectional view of the crank assembly 900 of a leaflet puncture and slitting device 100 in the straight or piercing configuration, according to aspects of the present disclosure. Visible are the crank 140, crank lever 145, top link 760, bottom link 770, and bottom slider 780. The crank 140 rotates around a hub 910. The bottom link 770 is rotatably attached to the crank 140 by a first pivot pin 920, and is rotatably attached to the bottom slider 780 by a second pivot pin 930. The bottom link 770 also includes a recessed locking feature 940 that, in the straight or piercing configuration, wraps partway around the hub 910, such that a line 950 connecting the first pivot pin 920 to the second pivot pin 930 is offset from a line 960 connecting the hub 910 to the second pivot pin 930 by an offset 970. In an example, the offset 970 may be about 5 degrees, although other offsets both larger and smaller may be used instead or in addition. The offset 970 and the recessed locking feature 940, in combination, create a bi-stable configuration such that when the crank 140 is in the piercing configuration, it will tend to stay that way, unless the crank lever 145 is moved backward (e.g., proximally) with a force of, e.g., about 2 lbf.

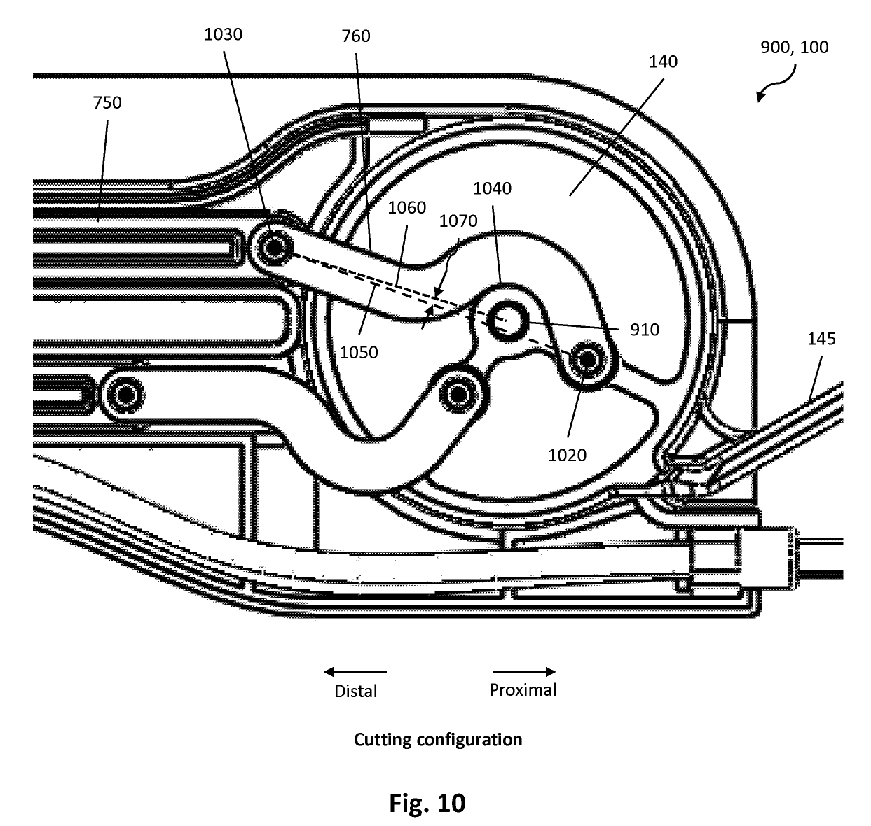

FIG. 10 is a close-up, side cross-sectional view of the crank assembly 900 of a leaflet puncture and slitting device 100 in the hooked or cutting configuration, according to aspects of the present disclosure. Visible are the crank 140, crank lever 145, top link 760, bottom link 770, top slider 750, and hub 910. The top link 760 is rotatably attached to the crank 140 by a first pivot pin 1020, and is rotatably attached to the top slider 750 by a second pivot pin 1030. The top link 760 also includes a recessed locking feature 1040 that, in the hooked or cutting configuration, wraps partway around the hub 910, such that a line 1050 connecting the first pivot pin 1020 to the second pivot pin 1030 is offset from a line 1060 connecting the hub 910 to the second pivot pin 1030 by an offset 1070. In an example, the offset 1070 may be about 5 degrees, although other offsets both larger and smaller may be used instead or in addition. The offset 1070 and the recessed locking feature 1040, in combination, create a bi-stable configuration such that when the crank 140 is in the hooked or cutting configuration, it will tend to stay that way, unless the crank lever 145 is moved forward (e.g., distally) with a force of, e.g., about 2 lbf.

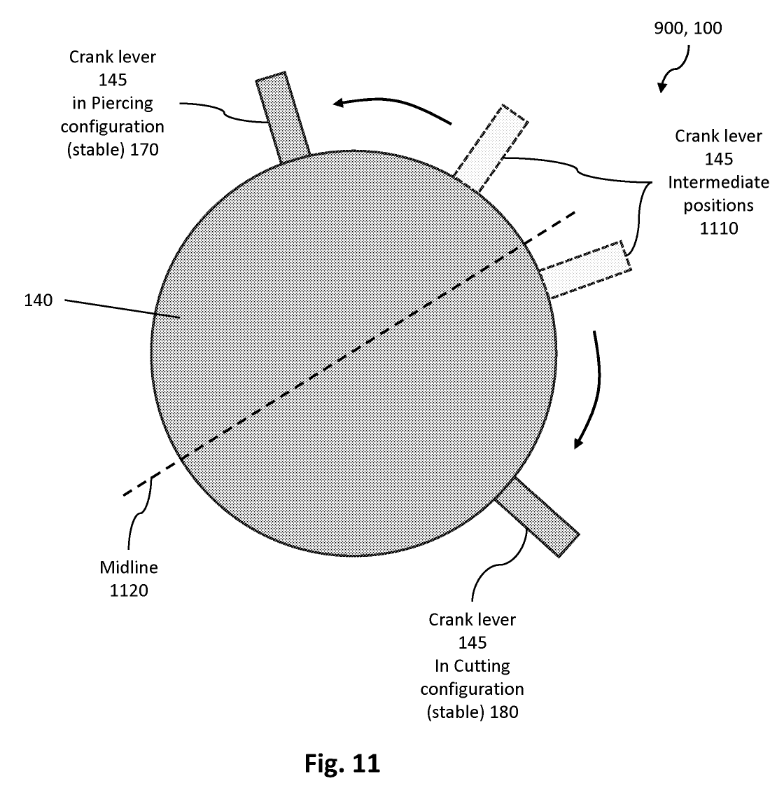

FIG. 11 is a schematic, diagrammatic, side view of the crank assembly 900 of a leaflet puncture and slitting device 100, showing a bi-stable configuration, according to aspects of the present disclosure. Visible are the crank 140 and crank lever 145. When the crank lever 145 is in the piercing configuration 170, its position is stable, as described above, and requires a force of e.g., approximately 2 lbf in a proximal or clockwise direction to move it out of this position. Similarly, when the crank lever 145 is in the cutting configuration 180, its position is also stable, and also requires a force of e.g., approximately 2 lbf in a counterclockwise or distal direction to move from that position.

However, when the crank lever 145 is in an intermediate position 1110, it will move automatically to the nearest stable position 170 or 180. For example, stable position 170 (piercing/straight configuration) can be a minimum crank angle θ and the stable position 180 (cutting/hooked configuration) can be a maximum crank angle θ (or vice versa). For example, relative to a midline 1120 (e.g., a mid-rotation crank angle θ), if the crank lever 145 is in an intermediate position 1110 closer to the cutting position 180 than to the piercing position 170, then the crank lever 145 will move to the cutting position 180. Similarly, relative to the midline 1120, if the crank lever 145 is in an intermediate position 1110 closer to the piercing position 170, then the crank lever 145 will automatically move to the piercing position 170. This bi-stable configuration allows the device to be held by a user (e.g., a heart surgeon or other clinician) without the application of constant force on the crank lever 145. Rather, the user can place the crank lever 145 in either the piercing configuration 170 or the cutting configuration 180, and it will stay that way until a force of, e.g., approximately 2 lbf is applied.

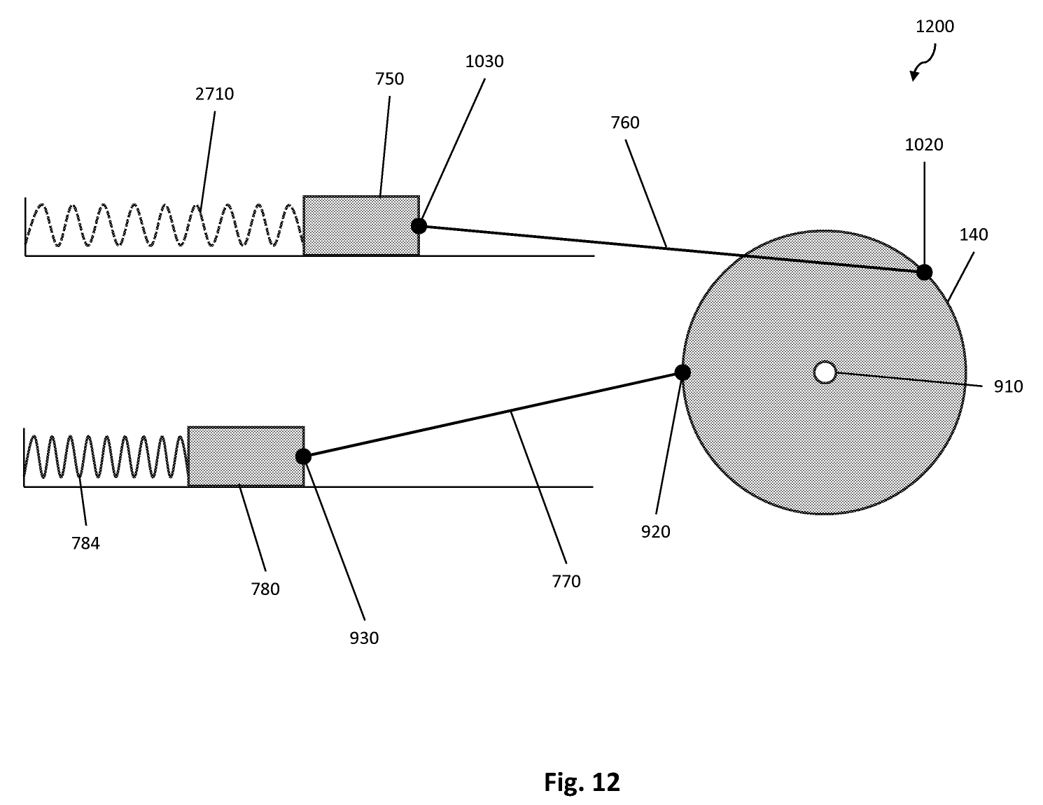

FIG. 12 is a schematic, diagrammatic, side-view representation of a self-locking pullwire actuator 1200, according to aspects of the present disclosure. Visible are the pullwire 2710 (modeled here as a spring), top slider 750, pivot pin 1030, top link 760, pivot pin 1020, crank 140, hub 910, pivot pin 920, bottom link 770, pivot pin 930, bottom slider 780, and counterbalance spring 784. In the example shown in FIG. 12, the self-locking pullwire actuator 1200 is in an unstable intermediate state in between the stable straight (piercing) and the stable hooked (cutting) configurations. In particular, the self-locking pullwire actuator 1200 is closer to the straight (piercing) configuration, and therefore unless a force is applied to the crank 140 (e.g., via the crank handle, not shown), the crank will rotate counterclockwise until the top slider is at the most distal end of its travel, and the bottom slider is at the most proximal end of its travel.

However, if the crank were rotated clockwise until the pivot pin 1020 was below the hub 910, then the self-locking pullwire actuator 1200 would be closer to the hooked or cutting configuration, and unless a force were applied to the crank 140 in a counterclockwise direction, the crank 140 would automatically rotate clockwise into the hooked or cutting configuration.

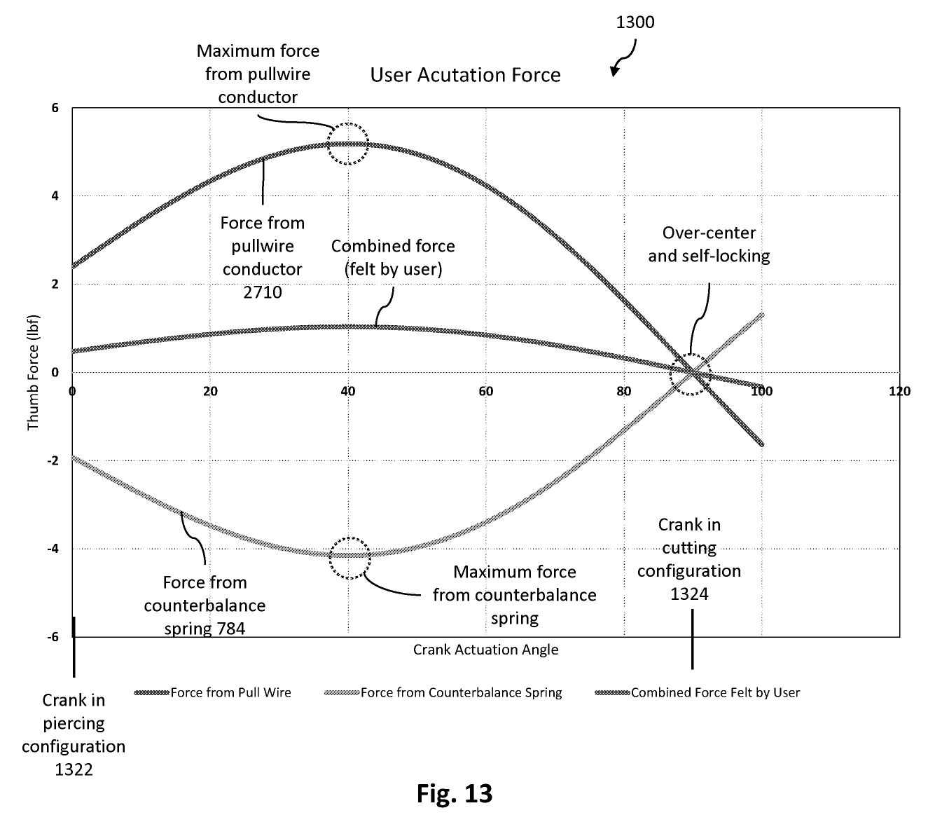

FIG. 13 is a graphical representation 1300 of the forces acting on a self-locking pullwire actuator, according to aspects of the present disclosure. The graphical representation includes a Y-axis 1310 measuring the value of force (units of lbf), and an x-axis 1320 representing the crank actuation angle q. Illustrated are the force from the pullwire conductor 2710, the force from the counterbalance spring 784, and the combined or net force (felt by the user). The parabola shape of the forces relates to the mechanical advantage of the crank slider mechanism. The crank actuation angle q is zero degrees when the crank is in the piercing/straight configuration and ninety degrees when the crank is in the cutting/hooked configuration. The zero degrees to ninety degrees values are exemplary and other ranges of values, both larger and smaller are contemplated. As the crank is rotated (crank actuation angle q increases), the force from the pullwire conductor 2710 and the force from the counterbalance spring 784 increases in magnitude. The forces are opposing, which is indicated by the magnitude of the force from the pullwire conductor 2710 increasing the positive direction (up along y axis) and the magnitude of the force from the counterbalance spring 784 increasing in in the negative direction (down along y axis). The maximum force from the pullwire 2710 occurs near the halfway point in the total travel/rotation of the crank. The maximum force from the counterbalance spring 784 occurs at approximately the same point as the maximum force from the pullwire 2710. Because of that, the combined force felt by the user does not change significantly through the total travel/rotation of the crank (e.g., generally remains between 0 lbf and 2 lbf). Going over center and self-locking is illustrated by the lines crossing over the zero force.

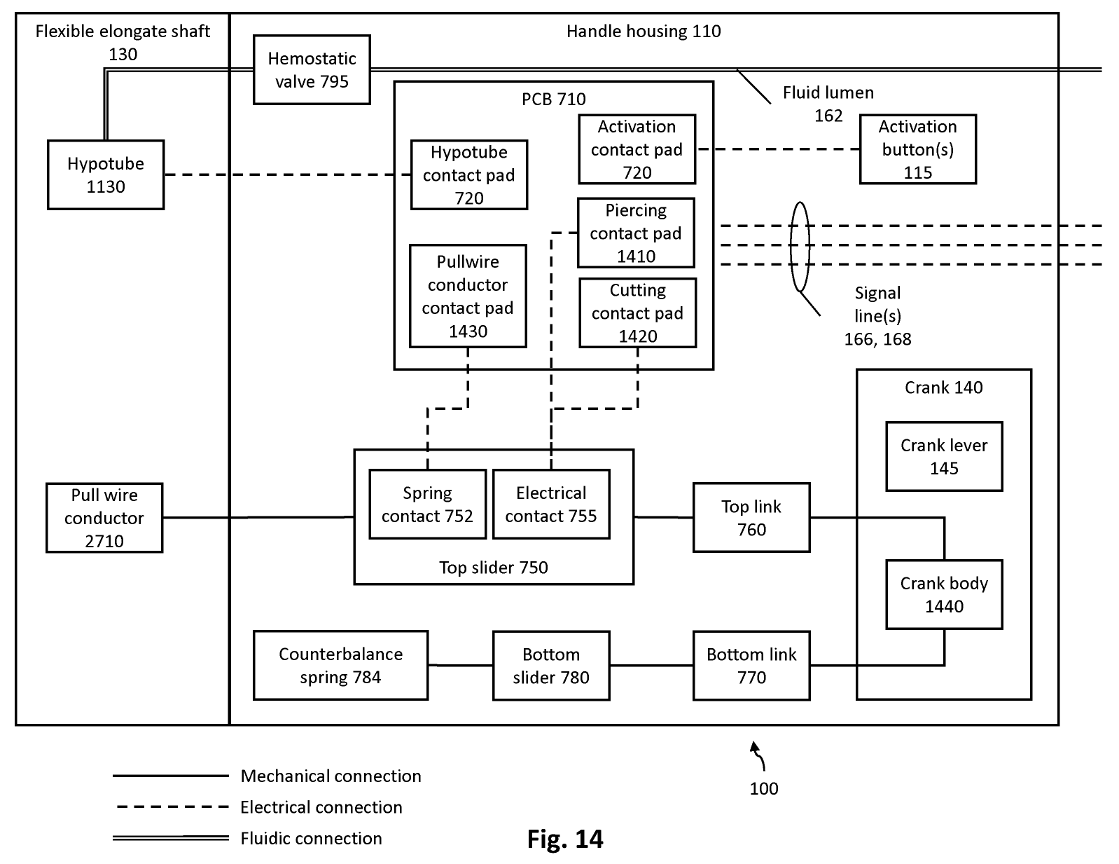

FIG. 14 is a schematic, diagrammatic representation, in block diagram form, of an example leaflet puncture and slitting device 100 that incorporates a self-locking pullwire actuator, according to aspects of the present disclosure. Inside of the handle 110 are components forming an actuation mechanism (e.g., pullwire 2710, coupling 740, slider 750, link 760, crank 140, crank lever 145, spring 784, slider 780, and/or link 770) for transitioning the distal portion of the device between two differently-shaped configurations (e.g., straight/piercing configuration and hooked/cutting configuration).

A printed circuit board (PCB) 710 includes an electrode activation contact pad 720 that can be activated by the button 115. When the electrode activation contact pad 720 is activated, then either the PCB 710 delivers electrical energy (RF energy) as current and/or voltage to the piercing electrode (via the pullwire conductor 2710, the pullwire conductor contact pad 1430 of the PCB 7110, and the spring contact 752 of the slider 750), when the electrical contact 755 of the slider 750 contacts the piercing contact pad 1410 (when the slider 750 is positioned as an FIG. 7) or the PCB 710 delivers electrical energy (RF energy) as current and/or voltage to the cutting electrode (via the hypotube 1130 and the hypotube contact pad 720 of the PCB 710), when the electrical contact 755 contacts the cutting contact pad 1420 (when the slider 750 is positioned as an FIG. 8). The PCB 710 receives electrical signals (e.g., RF energy and/or ground) via the signal lines 166 and/or 168.

The top link 760 is mechanically connected to the pullwire 2710 and to the crank body 1440, which is actuated by the crank lever 145 of the crank 140. The crank body 1440 is also mechanically connected to the bottom link 770, which drives the bottom slider 780 to push and/or pull on the counterbalance spring 784.

In the example shown in FIG. 14, a hemostatic valve 795 is in fluid communication with the hypotube 1130, for delivery of an electrically insulating fluid (e.g., a mixture of dextrose and water or dextrose and saline) that prevents electrical energy from the cutting electrode from propagating through surrounding body fluids such as blood. The hemostatic valve 795 can receive the fluid via fluid lumen 162.

Furthermore, the self-locking pullwire actuator mechanism can be applied with any catheter-based or other intraluminal flexible, elongate device that requires a high force and extended travel pull-wire actuation. One example is a leaflet resection (e.g., heart valve leaflet puncturing and/or slitting device), used in the treatment of heart valve related diseases. Aspects of the present disclosure can be applied to endoscopic, laparoscopic, intravascular, intraluminal, and/or robotic procedures.

As described herein, a handle mechanism for intraluminal devices addresses the challenges of high-force, extended-travel pullwire activation with minimal user effort. The actuation mechanism includes a dual crank-slider mechanism housed within the device handle. This mechanism utilizes a pair of balanced crank-slider assemblies working in tandem to provide mechanical advantage, reducing the force required from the user to activate the pullwire.

An aspect of the actuation mechanism is its self-locking over-center design. Each crank-slider assembly is engineered to move past its center point at the end of its travel, creating a stable locked position that prevents unintended release of the pullwire tension. This design ensures that the device’s distal tip remains securely in one of its two intended configurations (e.g., straight configuration or hooked configuration) without requiring continuous user input.

The mirrored arrangement of the two crank-slider mechanisms results in a bi-stable configuration. This means the system automatically biases towards either end of its range of motion, providing tactile feedback to the user and ensuring precise control over the device's positioning. To further enhance usability, the mechanism incorporates a counterbalance spring in the reverse mechanism. This spring offsets the force exerted by the pullwire, significantly reducing the effort required for user activation.

The combination of these features allows the handle to achieve extended output travel and generate high output forces while maintaining an ergonomic and easy-to-use design. This makes the device particularly suitable for applications requiring precise control and sustained positioning in minimally invasive procedures. The compact nature of the mechanism also allows for a sleek handle design that doesn't compromise on functionality or user comfort.

In an exemplary aspect, an intraluminal device includes a flexible elongate shaft configured to be positioned within a body lumen of a patient; and a pullwire disposed within the flexible elongate shaft and configured to transition a distal portion of the flexible elongate shaft between a first configuration and a second configuration; and a handle coupled to a proximal portion of the flexible elongate shaft, wherein the handle comprises an actuation mechanism coupled to the pullwire and configured to: transition the distal portion between the first configuration and the second configuration; and maintain the distal portion in the first configuration or in the second configuration without continuous input by a user.

In some aspects, the actuation mechanism is configured to: increase a tension on the pullwire to transition the distal portion from the first configuration to the second configuration; and decrease the tension on the pullwire to transition the distal portion from the second configuration to the first configuration. In some aspects, the actuation mechanism comprises a crank body and a crank lever coupled to the crank body, and rotation of the crank lever by the user is configured to transition the distal portion between the first configuration and the second configuration. In some aspects, the actuation mechanism further comprises: a top link coupled to the crank body; and a bottom link coupled to the crank body. In some aspects, the actuation mechanism is configured to maintain the distal portion in the first configuration or in the second configuration without the continuous input based on a first over-center position of the top link or and a second over-center position of the bottom link. In some aspects, the actuation mechanism further comprises: a top slider coupled to the top link; and a bottom slider coupled to the top link. In some aspects, the actuation mechanism further comprises a counterbalance spring coupled to the bottom slider, and the top slider is coupled to the pullwire. In some aspects, the counterbalance spring is configured to offset a force exerted by the pullwire and reduce a user input force for rotation of the crank lever. In some aspects, the actuation mechanism is configured to automatically transition to either the first configuration or the second configuration. In some aspects, the actuation mechanism is configured to transition to: the first configuration when the crank lever is rotated past a predetermined angle in a clockwise direction; and the second configuration when the crank lever is rotated past the predetermined angle in a counterclockwise direction. In some aspects, the intraluminal device further comprises a circuit board disposed within the handle. In some aspects, the intraluminal device further comprises a first electrode, and the circuit board comprises a first contact pad associated with energizing the first electrode when the distal portion is in the first configuration. In some aspects, the intraluminal device further comprises a second electrode, and the circuit board further comprises a second contact pad associated with energizing the second electrode when the distal portion is in the second configuration. In some aspects, the handle includes an activation button disposed on the handle and configured to selectively energize the first electrode when the distal portion is in the first configuration or the second electrode when the distal portion is in the second configuration. In some aspects, the first configuration comprises a straight shape and the second configuration comprises a hooked shape.

Block diagrams are provided herein for exemplary purposes; a person of ordinary skill in the art will recognize myriad variations that nonetheless fall within the scope of the present disclosure. Block diagrams may show a particular arrangement of components, modules, services, steps, processes, or layers, resulting in a particular flow of data, signals, energy, forces, or materials. It is understood that some embodiments of the systems disclosed herein may include additional components, that some components shown may be absent from some embodiments, and that the arrangement of components may be different than shown, resulting in different flows while still performing the methods described herein.

The logical operations making up the aspects of the technology described herein are referred to variously as operations, steps, objects, elements, components, or modules. Furthermore, it should be understood that these may occur, or be arranged or performed, in any order, unless explicitly claimed otherwise or a specific order is inherently necessitated by the claim language. It should further be understood that the described technology may be employed in single-use and multi-use devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader’s understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word "comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intraluminal device comprising:
a flexible elongate shaft configured to be positioned within a body lumen of a patient;
a pullwire disposed within the flexible elongate shaft and configured to transition a distal portion of the flexible elongate shaft between a first configuration and a second configuration; and
a handle coupled to a proximal portion of the flexible elongate shaft,
wherein the handle comprises an actuation mechanism coupled to the pullwire and configured to:
transition the distal portion between the first configuration and the second configuration; and
maintain the distal portion in the first configuration or in the second configuration without continuous input by a user.

2. The apparatus of claim 1, wherein the actuation mechanism is configured to:
increase a tension on the pullwire to transition the distal portion from the first configuration to the second configuration; and
decrease the tension on the pullwire to transition the distal portion from the second configuration to the first configuration.

3. The apparatus of claim 1,
wherein the actuation mechanism comprises a crank body and a crank lever coupled to the crank body,
wherein rotation of the crank lever by the user is configured to transition the distal portion between the first configuration and the second configuration.

4. The apparatus of claim 3, wherein the actuation mechanism further comprises:
a top link coupled to the crank body; and
a bottom link coupled to the crank body.

5. The apparatus of claim 1, wherein the actuation mechanism is configured to maintain the distal portion in the first configuration or in the second configuration without the continuous input based on a first over-center position of the top link or and a second over-center position of the bottom link.

6. The apparatus of claim 5, wherein the actuation mechanism further comprises:
a top slider coupled to the top link; and
a bottom slider coupled to the top link.

7. The apparatus of claim 6,
wherein the actuation mechanism further comprises a counterbalance spring coupled to the bottom slider,
wherein the top slider is coupled to the pullwire.

8. The apparatus of claim 7, wherein the counterbalance spring is configured to offset a force exerted by the pullwire and reduce a user input force for rotation of the crank lever.

9. The apparatus of claim 3, wherein the actuation mechanism is configured to automatically transition to either the first configuration or the second configuration.

10. The apparatus of claim 9, wherein the actuation mechanism is configured to transition to:
the first configuration when the crank lever is rotated past a predetermined angle in a clockwise direction; and
the second configuration when the crank lever is rotated past the predetermined angle in a counterclockwise direction.

11. The apparatus of claim 1, wherein the intraluminal device further comprises a circuit board disposed within the handle.

12. The apparatus of claim 11,
wherein the intraluminal device further comprises a first electrode,
wherein the circuit board comprises a first contact pad associated with energizing the first electrode when the distal portion is in the first configuration.

13. The apparatus of claim 12,
wherein the intraluminal device further comprises a second electrode,
wherein the circuit board further comprises a second contact pad associated with energizing the second electrode when the distal portion is in the second configuration.

14. The apparatus of claim 13, wherein the handle further comprises an activation button disposed on the handle and configured to selectively energize the first electrode when the distal portion is in the first configuration or the second electrode when the distal portion is in the second configuration.

15. The apparatus of claim 1, wherein the first configuration comprises a straight shape and the second configuration comprises a hooked shape.
